(19) United States
(12) Patent Application Publication
(10) Pub. No.: US 2008/0176928 A1
(43) Pub. Date: Jul. 24, 2008

(54) Avermectin/hydrocortisone compositions for treating afflictions of the skin, E.G., rosacea

(75) Inventors: Alexandre Kaoukhov, Juan Les Pins (FR); Colette Pernin, Nice (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 12/000,186

(22) Filed: Dec. 10, 2007

Related U.S. Application Data
(63) Continuation of application No. PCT/FR2006/001299, filed on Jun. 8, 2006.

(30) Foreign Application Priority Data
Jun. 10, 2005 (FR) 0505919

(51) Int. Cl. A61K 31/352; A61P 17/00

(52) U.S. Cl. 514/453

(57) ABSTRACT

Pharmaceutical/dermatological compositions containing at least one avermectin compound, e.g., ivermectin and hydrocortisone or salt, ester or derivative thereof, are useful for treating afflictions of the skin, especially rosacea.

### CROSS-REFERENCE TO PRIORITY/PCT APPLICATIONS

This application claims priority under 35 U.S.C. § 119 of FR 05/05919, filed Jun. 10, 2005, and is a continuation of PCT/FR 2006/001299, filed Jun. 8, 2006, and designating the United States (published in the French language on Dec. 14, 2006 as WO 2006/131651 A2; the title and abstract were published in English), each hereby expressly incorporated by reference in its entirety and each assigned to the assignee hereof.

### BACKGROUND OF THE INVENTION

1. Technical Field of the Invention

The present invention relates to pharmaceutical compositions, and especially dermatological compositions, for treating skin conditions and afflictions, and especially for treating rosacea (formerly known as acne rosacea).

In particular, the present invention relates to pharmaceutical compositions, especially dermatological compositions, comprising, formulated into a physiologically acceptable medium, at least one compound of the avermectin family and hydrocortisone.

2. Description of Background and/or Related and/or Prior Art

Rosacea is a chronic inflammatory dermatitis that mainly affects the median part of the face and the eyelids of certain adults. It is characterized by telangiectatic erythema, dryness of the skin, papules and pustules.

Conventionally, rosacea develops in adults from the ages of 30 to 50; it more frequently affects women, although the condition is generally more severe in men.

Despite its former name, acne rosacea is not a condition of the pilosebaceous follicles like juvenile acne, but a primitively vascular condition whose inflammatory stage lacks the cysts and comedones characteristic of common acne.

The aetiology of rosacea is still poorly understood, although many theories have been advanced. The most common hypothesis is based on the characteristic presence of the parasite Demodex folliculorum in the case of patients suffering from rosacea. This organism is absent in the other forms of acne such as common acne. Other factors have been described as possibly contributing towards the development of rosacea, such as hormonal factors and especially endocrine factors, climatic and immunological factors, and bacterial factors via the presence of Helicobacter pylori, a bacterium associated with gastrointestinal disorders.

Rosacea develops in four stages over several years, in spasms aggravated by variations in temperature, alcohol, spices, exposure to sunlight and emotions. The various stages of the disease are the following:

Stage 1: stage of erythema episodes. The patients have erythrosis spasms due to the sudden dilation of the arterioles of the face, which then take on a congestive, red appearance. These spasms are caused by the emotions, meals and temperature changes.

Stage 2: stage of couperosis, i.e., of permanent erythema with telangiectasia. Certain patients also have oedema on the cheeks and the forehead.

Stage 3: inflammatory stage with appearance of inflammatory papules and pustules, but without affecting the sebaceous follicles and thus with absence of cysts and comedones.

Stage 4: rhinophyma stage. This late phase essentially affects men.

The patients have a bumpy, voluminous red nose with sebaceous hyperplasia and fibrous reordering of the connective tissue.

Conventionally, rosacea is treated orally or topically with antibiotics such as tetracyclines, erythromycin or clindamycin, but also with vitamin A, salicylic acid, anti-fungal agents, steroids, metronidaxzole (an anti-bacterial agent) with anti-infectious agents such as benzoyl peroxide, or with isotretinoin in severe cases or even with hydrocortisone.

Hydrocortisone is known in the prior art as a glucocorticoid, anti-inflammatory agent, immunosuppressant, dermocorticoid, mineralocorticoid and anti-allergic agent. Hydrocortisone is a mixed anti-inflammatory agent that acts in primary and secondary inflammation at the acute stage of inflammation. It stabilizes the lysosomal membrane during the proteolytic catabolic phase and increases the capillary tonus during the exudative rectional phase. Hydrocortisone acts at the granuloma stage during the proliferative anabolic phase of repair, with an inhibition of fibroblast proliferation, mucopolysaccharide synthesis and collagen formation.

U.S. Pat. No. 5,952,372 also describes a method for treating rosacea using ivermectin orally or topically in order to reduce and eliminate the parasite Demodex folliculorum present on the skin of patients.

Ivermectin belongs to the avermectin family, a group of macrocyclic lactones produced by the bacterium Streptomyces avermitilis (Reynolds JEF (Ed) (1993) Martindale. The Extra Pharmacopoeia., 29th Edition. Pharmaceutical Press, London).

The avermectins especially include ivermectin, invermectin, avermectin, abamectin, doramectin, eprinomectin and selamectin.

Ivermectin is known in the prior art for its anti-parasitic and anthelmintic properties. The anti-parasitic activity is thought to be due to the opening of a chlorine channel in the membrane of the neurons of the parasite under the effect of an increased release of the neuromediator GABA (gamma-aminobutyric acid), inducing neuromuscular paralysis that may lead to the death of certain parasites. Ivermectin also interacts with other chlorine channels, especially those dependent on the neuromediator GABA (gamma-aminobutyric acid).

Ivermectin is conventionally administered in the dermatological treatment of endoparasitic manifestations such as onchocerciasis and myiasis. U.S. Pat. No. 6,133,310 describes the use of ivermectin in the treatment of rosacea in order to reduce and eliminate the parasite Demodex folliculorum present on the skin of patients.

However, these treatments have drawbacks such as irritation and intolerance phenomena, especially when they are administered for a prolonged period. On the other hand, these treatments are only suppressive and not curative, acting especially on the pustulous spasms occurring during the inflammatory stage. Furthermore, hydrocortisone frequently induces a rebound effect after the end of the treatment.

Considering the chronic nature of rosacea, the ideal treatment requires prolonged use, in a safe and effective manner. Taking the foregoing into account, need thus exists for compositions that show improved efficacy in the treatment of rosacea and that do not have the side effects described in the prior art.

Need especially exists for compositions that provide greater tolerance to the active principles thereof, while at the same time reducing their side effects.

### SUMMARY OF THE INVENTION

Accordingly, the present invention features pharmaceutical compositions, especially dermatological compositions, comprising, formulated into a physiologically acceptable medium, at least one compound of the avermectin family and hydrocortisone.

The subject compositions comprising the particular combination hereof reduce or eliminate the rebound effect typically encountered at the end of treatment with hydrocortisone.

The term “physiologically acceptable medium” means any medium that is compatible with the skin, mucous membranes and/or the integuments.

The hydrocortisone according to the invention may be used in unmodified form, or alternatively in the form of a salt with a pharmaceutically acceptable base, or else in the form of an ester or of a derivative. The term “esters” especially means 21-succinate, 21-acetate, 21-butyrate, 17-butyrate, 17-valerate, 21 β-cyclopentanepropionate (or cypionate), aceponate or even buteprate. The term “derivatives” means compounds that differ from hydrocortisone by substitution, addition or removal of one or more chemical groups and that have substantially the same activity.

Thus, this invention features pharmaceutical compositions, especially dermatological compositions, comprising, formulated into a physiologically acceptable medium, at least one compound of the avermectin family, and at least one compound selected from among hydrocortisone and salts, esters and derivatives thereof.

The present invention preferentially features pharmaceutical compositions, especially dermatological compositions, comprising, formulated into a physiologically acceptable medium, at least ivermectin and hydrocortisone.

This invention also features compositions formulated as medicaments for preventing and/or treating a skin condition.

Such compositions are especially for topical application.

The invention and the advantages resulting therefrom will become more apparent from the description which follows.

### DETAILED DESCRIPTION OF BEST MODE AND SPECIFIC/PREFERRED EMBODIMENTS OF THE INVENTION

The compounds of the avermectin family according to the present invention especially include invermectin, ivermectin, avermectin, abamectin, doramectin, eprinomectin and selamectin. The compound of the avermectin family is preferentially ivermectin.

In the compositions according to the invention, the said compound of the avermectin family is present in concentrations of from 0.001% to 10% by weight and preferably from 0.01% to 5% by weight relative to the total weight of the composition.

In the compositions according to the invention, the hydrocortisone and the salts, esters and/or derivatives thereof is/are present in concentrations of from 0.01% to 30% by weight and preferably from 0.01% to 15% by weight and particularly preferably from 0.01% to 5% by weight relative to the total weight of the composition.

Herein, unless otherwise specified, it is understood that when concentration ranges are given, they include the upper and lower limits of the said range.

Advantageously, the compositions of the invention comprise, other than the at least one compound of the avermectin family and hydrocortisone, at least one other therapeutic agent capable of increasing the efficacy of the treatment. Exemplary such agents include antibiotics, anti-bacterial agents, anti-viral agents, anti-parasitic agents, anti-fungal agents, anaesthetics, analgesics, anti-allergic agents, retinoids, free-radical scavengers, anti-pruritic agents, keratolytic agents, anti-seborrhoeic agents, anti-histamines, sulfides, and immunosuppressant or anti-proliferative products, or a mixture thereof.

The compositions according to the invention may also comprise any adjuvant usually employed in cosmetics and dermatology that is compatible with the said compound of the avermectin family and hydrocortisone. Especially exemplary are chelating agents, antioxidants, sunscreens, preservatives, fillers, electrolytes, humectants, dyes, common mineral or organic acids or bases, fragrances, essential oils, cosmetic active agents, moisturizers, vitamins, essential fatty acids, sphingolipids, self-tanning compounds, calmatives and skin-protecting agents, pro-penetrating agents and gelling agents, or a mixture thereof. These adjuvants, and the concentrations thereof, should be such that they do not adversely affect the advantageous properties of the mixture according to the invention. These additives may be present in the composition in a proportion of from 0% to 20% by weight and preferably from 1% to 10% by weight relative to the total weight of the composition.

Exemplary preservatives include benzalkonium chloride, phenoxyethanol, benzyl alcohol, diazolidinylurea and parabens, or mixtures thereof.

Humectants that are exemplary include glycerol and sorbitol.

Exemplary chelating agents include ethylenediaminetetraacetic acid (EDTA) and also derivatives or salts thereof, dihydroxyethylglycine, citric acid and tartaric acid, or mixtures thereof.

Pro-penetrating agents that are exemplary include propylene glycol, dipropylene glycol, propylene glycol dipelargonate, lauryl glycol and ethoxydiglycol.

The compositions according to the invention are useful, whether in a regime or regimen, for treating and/or preventing rosacea.

According to a first embodiment of the invention, the subject compositions are useful for formulating medicaments for treating the skin and preferably for treating rosacea, common acne and seborrhoeic dermatitis and particularly preferably for treating rosacea.

The present invention also features the use of at least one compound of the avermectin family and of at least one compound selected from hydrocortisone, salts, esters and/or derivatives thereof for the formulation of pharmaceutical compositions, and especially dermatological compositions, for preventing and/or treating a skin condition.

The compositions according to the invention are pharmaceutical compositions, and especially dermatological compositions, which may be in any galenical form conventionally used for topical application and especially in the form of aqueous gels, and aqueous or aqueous-alcoholic solutions. By addition of a fatty or oily phase, it may also be in the form of dispersions of the lotion or serum type, emulsions of liquid or semi-liquid consistency of the milk type obtained by dispersing a fatty phase in an aqueous phase (O/W) or conversely (W/O), or suspensions or emulsions of soft, semi-liquid or solid consistency of the cream, gel or ointment type, or alternatively multiple emulsions (W/O/W or O/W/O), microemulsions, microcapsules, microparticles or vesicular dispersions of ionic and/or nonionic type, or wax/aqueous phase dispersions. These compositions are formulated according to the usual methods.

When the composition is in emulsion form, the proportion of the oily phase of the emulsion may range, for example, from 5% to 80% by weight and preferably from 5% to 50% by weight relative to the total weight of the composition. The oils, emulsifiers and co-emulsifiers used in the composition in emulsion form are selected from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

As fatty substances that may be used in the invention, exemplary are oils and especially mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols such as cetyl alcohol, fatty acids, waxes and gums, in particular silicone gums, may also be used as fatty substances.

As emulsifiers and co-emulsifiers according to the invention, exemplary are fatty acid esters of polyethylene glycol such as PEG-100 stearate, PEG-50 stearate and PEG-40 stearate; fatty acid esters of polyols such as glyceryl stearate, sorbitan tristearate and the oxyethylenated sorbitan stearates available under the trademark Tween 20 or Tween 60, for example; and mixtures thereof.

Examples of gelling agents include the polyacrylamide family such as the sodium acryloyldimethyltaurate copolymer/isohexadecane/polysorbate 80 mixture marketed under the trademark Simulgel™ 600 by SEPPIC, the polyacrylamide/C13-14 isoparaffin/Laureth-7 mixture, for instance the product marketed under the trademark Sepigel 305™ by SEPPIC, the family of acrylic polymers coupled to hydrophobic chains, such as the PEG-150/decyl/SMDI copolymer marketed under the trademark Aculyn 44™ (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)), and the family of modified starches such as the modified potato starch marketed under the trademark Structure Solanace™, or mixtures thereof.

The preferred gelling agents are derived from the polyacrylamide family, such as Simulgel 600™ or Sepigel 305™, or mixtures thereof.

The gelling agent as described above may be used in a concentration ranging from 0.1% to 15% and preferably from 0.5% to 5%.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A pharmaceutical/dermatological composition useful for treating and/or preventing afflictions of the skin, comprising thus effective amounts of at least one avermectin compound and at least one compound selected from among hydrocortisone and salts, esters and derivatives thereof, formulated into a physiologically acceptable medium therefor.

2. The pharmaceutical/dermatological composition as defined by claim 1, comprising at least one avermectin compound selected from the group consisting of invermectin, ivermectin, avermectin, abamectin, doramectin, eprinomectin and selamectin.

3. The pharmaceutical/dermatological composition as defined by claim 2, comprising ivermectin.

4. The pharmaceutical/dermatological composition as defined by claim 1, comprising from 0.001% to 10% by weight of said at least one avermectin compound relative to the total weight thereof.

5. The pharmaceutical/dermatological composition as defined by claim 1, comprising from 0.01% to 30% by weight of hydrocortisone or salt, ester or derivative thereof relative to the total weight of the composition.

6. The pharmaceutical/dermatological composition as defined by claim 1, formulated for topical application.

7. The pharmaceutical/dermatological composition as defined by claim 1, further comprising at least one active agent selected from the group consisting of antibiotics, anti-bacterial agents, anti-viral agents, anti-parasitic agents, anti-fungal agents, anaesthetics, analgesics, anti-allergic agents, retinoids, free-radical scavengers, anti-pruritic agents, keratolytic agents, anti-seborrhoeic agents, anti-histamines, sulfides, and immunosuppressant or anti-proliferative compounds.

8. The pharmaceutical/dermatological composition as defined by claim 1, further comprising at least one additive selected from the group consisting of chelating agents, antioxidants, sunscreens, preservatives, fillers, electrolytes, humectants, dyes, mineral or organic acids or bases, fragrances, essential oils, cosmetic active agents, moisturizers, vitamins, essential fatty acids, sphingolipids, self-tanning compounds, calmatives and skin-protecting agents, pro-penetrating agents and gelling agents, or a mixture thereof.

9. A regime or regimen for preventing and/or treating an affliction of the skin, comprising administering to a subject in need of such treatment, a thus effective amount of at least one avermectin compound and at least one compound selected from among hydrocortisone and salts, esters and derivatives thereof.

10. A regime or regimen for treating and/or preventing rosacea, common acne and/or seborrhoeic dermatitis, comprising administering to a subject in need of such treatment, a thus effective amount of at least one avermectin compound and at least one compound selected from among hydrocortisone and salts, esters and derivatives thereof.

11. A regime or regimen for treating rosacea, comprising topically applying onto the afflicted skin area of an individual in need of such treatment, a thus effective amount of at least one avermectin compound and at least one compound selected from among hydrocortisone and salts, esters and derivatives thereof.
